(19) United States
(12) Patent Application Publication
(10) Pub. No.: US 2005/0188998 A1
(43) Pub. Date: Sep. 1, 2005

(54) System, method, and apparatus for interchangeable decorations for ornamental sticks

(76) Inventor: Cynthia Ann Salas, Pflugerville, TX (US)

Correspondence Address: BRACEWELL & PATTERSON, LLP;IP DOCKETING, P.O. BOX 61389, HOUSTON, TX, 77208-1389, US

(21) Appl. No.: 11/063,780

(22) Filed: Feb. 23, 2005

Related U.S. Application Data
(60) Provisional application No. 60/548,955, filed on Mar. 1, 2004.

(51) Int. Cl. A61K 7/04; A45D 7/02

(52) U.S. Cl. 132/212

(57) ABSTRACT

A system for interchanging ornamental sticks and their decorative portions is disclosed. The ornamental sticks may be used as hair sticks or chop sticks. The decorative portions of the ornamental sticks are easily and readily interchanged with the structural support sticks to create many different designs. These designs may be quickly changed because of the unique retention mechanism utilized. For example, the retention mechanism may utilize threaded components or magnets to retain the decorative portions on the structural support sticks.

This is a non-provisional patent application that claims priority to and the benefit of provisional patent application U.S. Ser. No. 60/548,955, filed Mar. 1, 2004, and is incorporated herein by reference in its entirety.

### BACKGROUND OF THE INVENTION

1. Technical Field

The present invention relates in general to ornamental sticks and, in particular, to an improved system, method, and apparatus for interchanging ornamental sticks and the decorations on the ends of ornamental sticks.

2. Description of the Related Art

In the prior art, hair sticks for retaining hair styles in desired configurations are well known and have been used for centuries. Hair sticks are particularly well suited for helping women with long hair retain their hair in an upright configuration. Hair sticks have a rich history in many ancient cultures. Many references point to early Egyptian and Oriental origins that used hair adornments such as sticks, forks, combs, and spikes. In many cultures across a vast amount of history, women wore hair sculpture and adornments reflecting a person's individual status, social class, and the different stages in their lives.

Materials used for hair sticks throughout the world changed according to culture and time. Egyptians and tribes in the Savannah region traditionally used gold, elaborately decorated with gemstones, shells, and coral. Other cultures saw gold as an evil and forbidden material such as the Moorish tribes. The use of ivory was highly elite and limited only to royalty. Shells and coral which represented not only protection, but also fertility were common in hair adornments. In Native American, Celtic, and African cultures, bone was widely used in various types of hair forks and sticks, often elaborately carved and polished. The hair ornaments worn by Japanese geisha and maiko (apprentice geisha) throughout history were ornate hair pins which could be worn alone or in combination with other hair sticks in the more traditional Asian style.

Each of these styles utilizes a permanent construction wherein the stick or pin is permanently mounted to the decorative elements of the design. In order to change a style, the user must select and utilize a completely different hair stick, which requires the user to acquire a large assortment of designs. For the average user, such an assortment is too cumbersome and, in some cases, costly to maintain. Thus, an improved solution for flexibly and economically maintaining an assortment of hair stick designs would be desirable.

### SUMMARY OF THE INVENTION

One embodiment of a system, method, and apparatus for interchanging ornamental sticks and their decorative portions is disclosed. The ornamental sticks may be used as hair sticks or chop sticks. The decorative portions of the ornamental sticks are easily and readily interchanged with the structural support sticks to create many different designs. These designs may be quickly changed because of the unique retention mechanism utilized. For example, the retention mechanism may utilize threaded components or magnets to retain the decorative portions on the structural support sticks.

The foregoing and other objects and advantages of the present invention will be apparent to those skilled in the art, in view of the following detailed description of the present invention, taken in conjunction with the appended claims and the accompanying drawings.

### BRIEF DESCRIPTION OF THE DRAWINGS

So that the manner in which the features and advantages of the invention, as well as others which will become apparent are attained and can be understood in more detail, more particular description of the invention briefly summarized above may be had by reference to the embodiment thereof which is illustrated in the appended drawings, which drawings form a part of this specification. It is to be noted, however, that the drawings illustrate only an embodiment of the invention and therefore are not to be considered limiting of its scope as the invention may admit to other equally effective embodiments.

FIG. 1 is a side view of one embodiment of an ornamental stick constructed in accordance with the present invention;

FIG. 2 is an exploded view of the ornamental stick of FIG. 1 and is constructed in accordance with the present invention;

FIG. 3 is an enlarged sectional view of the ornamental stick of FIG. 1 and is constructed in accordance with the present invention; and

FIG. 4 is an enlarged sectional view of an alternate embodiment of the ornamental stick of FIG. 1 and is constructed in accordance with the present invention.

### DETAILED DESCRIPTION OF THE INVENTION

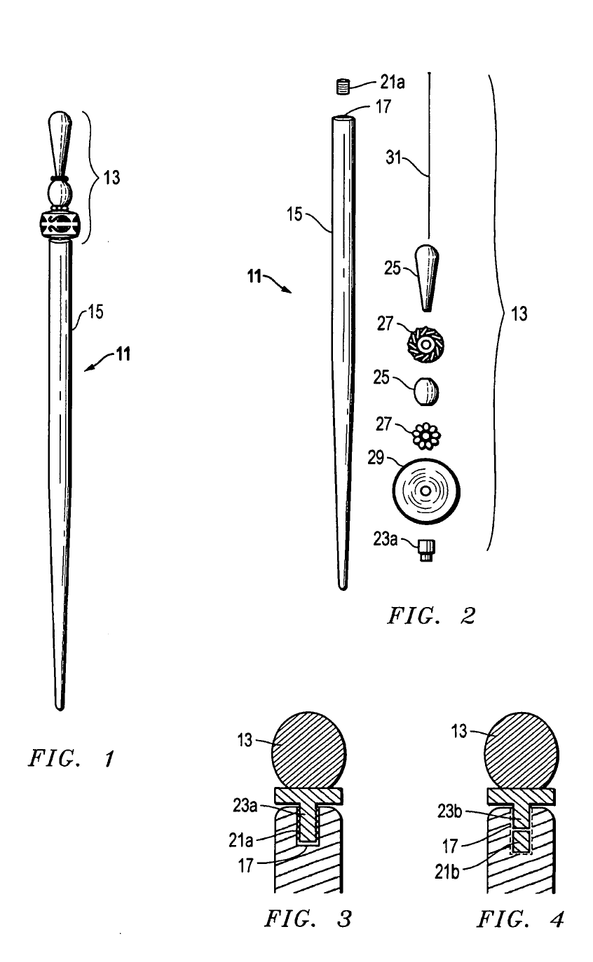

Referring to FIGS. 1-4, embodiments of a system, method, and apparatus for interchanging ornamental sticks 11 and their decorative portions 13 is disclosed. The ornamental sticks 11 have many different uses, but are ideally suited for use as hair sticks to decorate and retain hairstyles in a desired coiffure. In this application, the ornamental sticks may be used in pairs or as individual pieces. As another example, the ornamental sticks also may be used as chop sticks while dining. Ornamental sticks are also used for many other applications and known by many other names (e.g., hair stix, hair pins, etc.). The term “ornamental sticks” is used for illustration purposes only.

The decorative portions 13 of the ornamental sticks 11 are easily and readily interchanged with the structural support sticks 15 to create an infinite number of different designs. For example, the decorative portions 13 may comprise many different embellishments, toppers, decorator beads, etc. These designs may be quickly changed because of the unique retention mechanism utilized. Because the decorative portions 13 and support sticks 15 are interchangeable, a user is able to change the decorative portions 13 without having to carry around numerous support sticks 15. In one embodiment, the decorative portions 13 are pre-assembled in a permanent configuration or design. In this design, the beads, spacers, findings, discs, decorative elements, etc., that form a decorative portion 13 are not intended to be altered due to the way they are manufactured. However, in another embodiment, all of the elements (or sub-components thereof) comprising the decorative portions 13 of the ornamental sticks 11 are interchangeable.

To illustrate and describe the design and operation of interchangeable ornamental sticks 11, the following description of an ornamental stick 11 will start from the structural support stick 15 and work toward the decorative portion 13. The material used to form the structural support stick 15 many different materials, including bone and wood. These materials may be used in their natural state, and/or dyed, painted, or otherwise decorated. Other materials suitable for use as a stick 15 include metals such as silver, gold, or stainless steel, and plastic or acrylic in virtually any color. In one embodiment, the length of the stick 15 may range from about 4 to 8 inches, and may comprise a variety of forms (e.g., slightly widened on one end tapering down to a point). The sticks 15 also may be twisted, etched, carved, or otherwise artistically configured.

In the embodiment shown, each stick 15 has a small axial hole 17 on one end. The hole 17 may be drilled into the stick 15 or otherwise formed. The diameter of the hole 17 is slightly larger than a retention mechanism 21 that is placed inside the hole 17. In one embodiment, the retention mechanism 21 is secured to the stick 15 via adhesive bonding or other means.

For example, the retention mechanism 21 may comprise a threaded element 21a (FIG. 3) or a magnet 21b (FIG. 4). When a threaded element 21a (e.g., threaded female receptacle) is used, the axial depth of the hole 17 is approximately equal to the axial height of the threaded element 21a, such that the threaded element 21a is approximately flush with the outer surface of the stick 15 when it is mounted in hole 17. However, when a magnet 21b is used, the axial depth of the hole 17 is greater than the axial height of the magnet 21b, such that the magnet 21b is axially recessed within the hole 17 and spaced apart from the outer surface of the stick 15 when it is mounted in hole 17. In an alternate embodiment, the magnet 21b also may be flush-mounted to the exterior of the stick 15.

The retention mechanism 21 comprises one part of a two component clasp. The other part of the clasp (i.e., the engagement mechanism 23) is mounted to the decorative portion 13. For example, the engagement mechanism may comprise a threaded element 23a (FIG. 3) for engaging threaded element 21a, or a magnet 23b (FIG. 4) for engaging magnet 21b. The engagement mechanism 23 extends from the decorative portion 13 to insert into hole 17 and engage the retention mechanism 21. In this way, the decorative portion 13 is removably retained on the stick 15 to form a complete ornamental stick 11.

For example, threaded elements 21a and 23a threadingly engage, and magnets 21b and 23b magnetically engage (i.e., they are oppositely poled). The mechanical force provided by threaded elements 21a, 23a, and the magnetic force between magnets 21b, 23b is sufficient to adequately retain decorative portion 13 on stick 15 without being unduly burdensome to readily interchange other decorative portions 13 and sticks 15 to form other configurations of ornamental sticks 11. While utilizing either embodiment, the decorative portion 13 appears flush with stick 15 when mounted thereto such that the clasp is not visible. The components of the clasp may be formed from a variety of materials, including metals such as gold, sterling silver, gold-fillings, stainless steel, nickel, brass, copper, and other metals in gold plate and silver plate. The components of the clasp also can be made in several different shapes such as bullets, squares and rounds.

The decorative portion 13 may comprise, for example, beads 25, spacers 27, discs 29, etc. The pieces used to form the decorative portion 13 may be adhesively bonded, hammered, or otherwise joined together. These components may be from many different types of substances including metal, plastic (e.g., acrylic), glass, metalized materials, gold filled, pewter, porcelain, fiber optic, pearls, bone, fimo clay (i.e., a clay that can be baked and formed to a desired size and shape), crystal, glass foil, sterling silver-lined, gold sterling silver, gold plate, silver plate, copper, nickel, brass, etc. Other materials include: Austrian Crystal, Swarovski crystal, gemstones in their genuine and semi-precious state, including carved, metal plate, rondelles in silver and gold plate with and without rhinestones and crystals, gold plate, pewter, antique, bali, plastic/acrylic, porcelain, bone, seed glass, glass foil, sterling silver lined, pressed glass, natural shells, African, Swiss, Czech, Japanese, Bally, Indonesian, Malaysian, European, Indian, lamp work, cane glass, art glass, ivory, crow beads, tile beads, fire polished, rocaille glass seed, glass tube, glass & plastic bugle, wood, hematite, hemalyke, fiber optic, cloisonné, ceramic, dichroic beads, milleflori, tortoise, bead caps (used as a cup to hold a piece in place and give it a more professional look), pearls (i.e., plastic, glass, genuine, semi-precious, cultured), hand made lamp beads, hand blown and handmade pieces, imported pieces, etc. These pieces also come in a variety of forms such as squares, cylinders, balls, cones, barrel shapes, etc.

In addition, a finding 31 is used to secure the components of decorative portion 13 together. For example, finding 31 may comprise a head pin with a flat head. This piece can range from, for example, 1 to 3 inches in length. Finding 31 may be formed from a variety of materials, such as metals including gold plate, silver plate, gold, sterling silver, nickel, brass, copper and gold filled. An eye pin, which has a small loop at the end instead of the flat head, can also be used to string beads and metals. Using this loop type will give one the ability to hang charms from the loop using sterling silver, gold, nickel, copper, brass, bone, natural shells, plated gold and silver, pewter, crow beads, tile beads, plastic/acrylic, cloisonné, crystal, fiber optic, glass foil and sterling silver lined, ceramic, tortoise, hematite, cane glass, pressed glass, genuine and semi precious stones, pearls, milleflori, handmade, lamp worked, etc. The components of the decorative portion 13 may be joined together, with the exception of the two-piece clasp that interlocks. The finding 31 is trimmed and filed, if necessary, to fit just inside the mechanism piece and to prevent scratching.

In one illustrative embodiment, the present invention provides interchangeable ornamental sticks that comprise a plurality of tapered sticks, each having a longitudinal axis, a distal end, a proximal end located opposite the distal end, an axial blind hole formed in the proximal end, and a retention mechanism mounted in the axial blind hole. A plurality of decorative portions, each having ornamental features and an engagement mechanism, is used for engaging the retention mechanisms and removably retaining the decorative portions on the tapered sticks. The plurality of tapered sticks is completely and readily interchangeable with the plurality of decorative portions via respective ones of their retention mechanisms and engagement mechanisms. The ornamental sticks may be provided as hair sticks for decorating hair styles, as chop sticks for dining, or for other uses. The retention and engagement mechanisms may be secured to the tapered sticks and to the decorative portions, respectively, via adhesive bonding.

In one version, each of the retention mechanisms comprises a first threaded element, and each of the engagement mechanisms comprises a second threaded element that is threadingly engageable with individual ones of the first threaded elements. In this version, an axial depth of the axial blind holes is approximately equal to an axial length of the first threaded elements, such that the first threaded elements are approximately flush with outer surfaces of the tapered sticks. This version may be further modified by specifying the first threaded elements as female threads, and the second threaded elements as male threads and threadingly engage the first threaded elements.

In another version, each of the retention mechanisms comprises a first magnetic element, and each of the engagement mechanisms comprises a second magnetic element that magnetically engages individual ones of the first magnetic elements. In this version, an axial depth of the axial blind hole is greater than an axial length of the first magnetic element, such that the first magnetic element is axially recessed within the axial blind hole and is axially spaced apart from an outer surface of the tapered stick. In addition, the second magnetic element may axially insert into the axial blind hole to abut and magnetic engage the first magnetic element to retain the decorative portion on the tapered stick.

While the invention has been shown or described in only some of its forms, it should be apparent to those skilled in the art that it is not so limited, but is susceptible to various changes without departing from the scope of the invention.

1. An ornamental stick, comprising:
a tapered stick having a longitudinal axis, a distal end, a proximal end located opposite the distal end, an axial blind hole formed in the proximal end, and a retention mechanism mounted in the axial blind hole;
a decorative portion having ornamental features and an engagement mechanism for engaging the retention mechanism and removably retaining the decorative portion on the tapered stick; and
other ones of the tapered sticks and other ones of the decorative portions are readily interchangeable via respective retention mechanisms and engagement mechanisms.

2. An ornamental stick according to claim 1, wherein the ornamental stick is a hair stick for decorating hair styles.

3. An ornamental stick according to claim 1, wherein the ornamental stick is a chop stick for dining.

4. An ornamental stick according to claim 1, wherein the retention and engagement mechanisms are secured to the tapered stick and to the decorative portion, respectively, via adhesive bonding.

5. An ornamental stick according to claim 1, wherein the retention mechanism comprises a first threaded element, and the engagement mechanism comprises a second threaded element that threadingly engages the first threaded element.

6. An ornamental stick according to claim 5, wherein an axial depth of the axial blind hole is approximately equal to an axial length of the first threaded element, such that the first threaded element is approximately flush with an outer surface of the tapered stick.

7. An ornamental stick according to claim 5, wherein the first threaded element is a female thread, and the second threaded element is a male thread and threadingly engages the first threaded element.

8. An ornamental stick according to claim 1, wherein the retention mechanism comprises a first magnetic element, and the engagement mechanism comprises a second magnetic element that magnetically engages the first magnetic element.

9. An ornamental stick according to claim 8, wherein an axial depth of the axial blind hole is greater than an axial length of the first magnetic element, such that the first magnetic element is axially recessed within the axial blind hole and is axially spaced apart from an outer surface of the tapered stick.

10. An ornamental stick according to claim 9, wherein the second magnetic element axially inserts into the axial blind hole to abut and magnetic engage the first magnetic element to retain the decorative portion on the tapered stick.

11. A system of interchangeable ornamental sticks, comprising:
a plurality of tapered sticks, each having a longitudinal axis, a distal end, a proximal end located opposite the distal end, an axial blind hole formed in the proximal end, and a retention mechanism mounted in the axial blind hole;
a plurality of decorative portions, each having ornamental features and an engagement mechanism for engaging the retention mechanisms and removably retaining the decorative portions on the tapered sticks; and
the plurality of tapered sticks being completely and readily interchangeable with the plurality of decorative portions via respective ones of their retention mechanisms and engagement mechanisms.

12. A system according to claim 11, wherein the ornamental sticks are hair sticks for decorating hair styles.

13. A system according to claim 11, wherein the ornamental sticks are chop sticks for dining.

14. A system according to claim 11, wherein the retention and engagement mechanisms are secured to the tapered sticks and to the decorative portions, respectively, via adhesive bonding.

15. A system according to claim 11, wherein each of the retention mechanisms comprises a first threaded element, and each of the engagement mechanisms comprises a second threaded element that is threadingly engageable with individual ones of the first threaded elements;
an axial depth of the axial blind holes is approximately equal to an axial length of the first threaded elements, such that the first threaded elements are approximately flush with outer surfaces of the tapered sticks; and
the first threaded elements are female threads, and the second threaded elements are male threads and threadingly engage the first threaded elements.

16. A system according to claim 11, wherein each of the retention mechanisms comprises a first magnetic element, and each of the engagement mechanisms comprises a second magnetic element that magnetically engages individual ones of the first magnetic elements;
an axial depth of the axial blind holes is greater than an axial length of the first magnetic elements, such that the first magnetic elements are axially recessed within the axial blind holes and are axially spaced apart from outer surfaces of the tapered sticks; and
individual ones of the second magnetic elements axially insert into the axial blind holes to abut and magnetic engage the first magnetic elements to retain the decorative portions on the tapered sticks.

17. A method of interchanging components of ornamental sticks, the method comprising:
(a) providing a tapered stick having a longitudinal axis, an axial blind hole formed in an end, and a retention mechanism mounted in the axial blind hole;
(b) providing decorative portions, each having an engagement mechanism;
(c) removably joining one of the decorative portions to the tapered stick by engaging the engagement mechanism to the retention mechanism; and then
(d) exchanging another one of the decorative portions with said one of the decorative portions on the tapered stick to change an ornamental design thereof via respective the retention mechanism and the engagement mechanism.

18. A method according to claim 17, further comprising the step of securing the retention and engagement mechanisms to the tapered stick and to the decorative portions, respectively, via adhesive bonding.

19. A method according to claim 17, wherein the retention mechanism comprises a first threaded element, and each of the engagement mechanisms comprises a second threaded element that threadingly engages the first threaded element;
an axial depth of the axial blind hole is approximately equal to an axial length of the first threaded element, such that the first threaded element is approximately flush with an outer surface of the tapered stick; and
the first threaded element is female thread, and the second threaded elements are male threads and threadingly engage the first threaded element.

20. A method according to claim 17, wherein the retention mechanism comprises a first magnetic element, and each of the engagement mechanisms comprises a second magnetic element that magnetically engages the first magnetic element;
an axial depth of the axial blind hole is greater than an axial length of the first magnetic element, such that the first magnetic element is axially recessed within the axial blind hole and is axially spaced apart from an outer surface of the tapered stick; and
individual ones of the second magnetic elements axially insert into the axial blind hole to abut and magnetically engage the first magnetic element to retain the decorative portions on the tapered stick.
